(19) United States
(12) Patent Application Publication
(10) Pub. No.: US 2017/0281814 A1
(43) Pub. Date: Oct. 5, 2017

(54) Method for Cleaning and Lubricating Medical/Dental Devices for Sterilization

(71) Applicants: Michael Overmyer, Danville, KY (US); Thad J. Overmyer, Danville, KY (US)

(72) Inventors: Michael Overmyer, Danville, KY (US); Thad J. Overmyer, Danville, KY (US)

(21) Appl. No.: 15/449,283

(22) Filed: Mar. 3, 2017

Related U.S. Application Data
(60) Provisional application No. 62/390,442, filed on Mar. 30, 2016.

(51) Int. Cl. A61L 2/18; A61C 19/00; A61L 2/04; A61B 90/70

(52) U.S. Cl. CPC A61L 2/18; A61B 90/70; A61C 19/002; A61L 2/04; A61L 2202/24

(57) ABSTRACT

A method of cleaning and lubricating a detached device within a sealed heat sterilizable container during the process of heat sterilization by using a liquid admixture comprised of a water-alcohol-glycerin-chlorhexidine solution.

### CROSS REFERENCE TO RELATED APPLICATION

This non-provisional application is a continuation-in-part of my copending provisional application titled, “Solution for Cleaning and Lubricating Medical/Dental Devices for Sterilization” filed on Mar. 30, 2016 and given Ser. No. 62/390,442.

### BACKGROUND OF THE INVENTION

Devices used in the medical and dental fields for procedures require cleaning, lubrication and sterilization. The heat sterilization process of a device affects the life span of the device. When the device is exposed to body fluids or sources of contaminates, the device requires cleaning and possibly lubrication of the device prior to sterilization. A device with moving parts (internal or external) should be cleaned and lubricated prior to sterilization.

When a device is not cleaned properly, contaminates remaining on the device will create a build up of sterilized layers of contaminates. This build up of contaminates may affect the function, sterilization efficiency and the life span of the device. The lubrication and cleaning products should not interfere with the sterilizing process or the mechanics of the autoclave.

The following devices in the dental and medical fields with moving parts that need proper cleaning and lubrication are the following: contra angle, laser, handpiece (slow and high speed), hinged device, and all surgical devices or any device that contacts the body internally or externally.

The contra angle can be exposed to the patient's body fluids: saliva and blood, soft tissue, tooth structure particles, restorative material particles, material for cleaning teeth, bone spicules, polishing burrs containing grit and rubber particles. The internal geared and ball bearing mechanisms of the contra angle have an entryway for exposure to contaminates. These mechanisms are cleaned and lubricated prior to sterilization.

Presently, the three known methods of cleaning and lubricating the contaminated contra angle prior to sterilization are the following:
1. Apply a lubricant product into the head of the contra angle.
2. Immerse the head of the contra angle in a cleaning solution and then immerse the head of the contra angle in a lubricating solution.
3. Take the contra angle apart, which then is cleaned and lubricated.
   After the cleaning and lubricating process is performed, then the contra angle is bagged for sterilization.

The high-speed handpiece has a turbine that can operate up to speeds of 500,000 RPM's. Although the bearings are encapsulated, the turbine's high-speed rotation creates a vacuum (“suck in”), which allows contaminates to be conveyed into the bearings. Over a period of time without proper cleaning and lubrication, the turbine's life span is affected by the build up of contaminates and the sterilization of the contaminates. Contaminates that can be sucked into the turbine may be oral fluids, blood, tooth spicules, filling material, or bone spicules. Any of these contaminates can be detrimental to the life span of the turbine.

Some handpieces have two conduits focused at the handpiece head. One conduit is a water outlet. The other conduit is an air outlet. Together the pressurized air and water creates a spray. The spray cools and washes the work piece and the handpiece head. The spray creates an aerosol during procedures. The aerosol can contaminate the handpiece and the atmosphere.

The handpiece is cleaned by manual or automated devices. These devices introduce an air source into the air drive conduit of the handpiece. This pressurized air forces the contaminates from the internal parts of the handpiece. For lubrication of the handpiece, an oily product is introduced into the drive airline to lubricate bearings and the turbine by a pressurized air method. The pressurized contaminates and lubricants exit through the handpiece's exhaust conduit. The handpiece is placed in a sterilization bag and sterilized.

The lubricant within the handpiece conduits (drive airline and exhaust conduit) prevents the penetration of the steam, which creates improper sterilization of the handpiece. The steam and the lubricant are not miscible. Lubricants can become gooey after sterilization. This gooey substance builds up within the handpiece's turbine and bearings, which affects the life span of the handpiece. The gooey substance is expelled when the handpiece is functioning after sterilization.

The air conduit and water conduit that goes to the head of the handpiece is untreated. No pressurized air or lubrication is forced into these conduits. Any contaminates surrounding the orifice or within the conduit of the water outlet are baked on the orifice or in the conduit during sterilization. This build up of contaminates affects the air and water flow to the head of the handpiece.

The laser has a water and air conduit that is used to cool the work piece and/or the device during procedures.

Maintaining a clean water conduit and air conduit is important to maintain proper laser function.

Earlier in my U.S. Patents
1. U.S. Pat. No. 4,695,255 I disclosed a solution of about 25% to 70% (volume of water, about 5% to 35% of volume of potable alcohol such as ethanol and about 10% to 45% by volume of glycerin. This patent uses the solution for cooling and lubricating human hard tissue during power tool cutting.
2. U.S. Pat. No 5,443,385, I disclosed a solution of about 35% to 60% (volume of water, about 3% to 20% of volume of potable alcohol such as ethanol, about 40% to 50% by volume of glycerin, 0.01% to 2.0% by volume of chlorhexidine gluconate, 0.01 to 5% by volume of polyglycol and about 1 gram of methylcellulose per liter of solution. This solution was used to lubricate and disinfect prior to autoclaving.

### SUMMARY OF INVENTION

The invention provides a method of cleaning and lubricating a detached device partially immersed in a preferred solution that is contained in a sealed heat sterilizable container during the process of heat sterilization.

The sealed heat sterilizable container contains the 120 detached device and the preferred solution. This sealed container is placed in a heat source such as the steam autoclave. During the autoclaving process, the preferred solution and the preferred solution's vapors penetrate the device's parts and conduits. In respects to a dental handpiece, the preferred solution penetrates the bearings or gears to clean and lubricate. The vapors of the boiling preferred solution enter the water, air, exhaust, and drive-air conduits to clean and lubricate these conduits.

The preferred solution is formulated from the following ingredients:
i. About 50% to about 85% by volume of water
ii. About 5% to about 22% by volume of glycerin
iii. About 0.05% to about 0.20% by volume of chlorhexidine gluconate
iv. About 3% to about 15% by volume of alcohol

### DESCRIPTION OF PREFERRED EMBODIMENT

The components of the preferred solution used in this invention are described by the following terms:

The water base is filtered deionized water and the potable alcohol is preferably denatured ethanol. The glycerin is a commercial Kosher grade suitable for human consumption. Chlorhexidine gluconate is commercially available in liquid form as chlorhexidine.

The device within the sealed heat sterilizable container is partially immersed in the preferred solution during heating process such as a steam autoclave. While the autoclave performs, the preferred solution's temperature is increased. During the heating process, the preferred solution boils around the enclosed device to clean and lubricate. The increased temperature of the preferred solution vaporizes and penetrates the conduits and all other areas of the device.

The preferred solution is miscible with any remaining water within the handpiece conduit. Therefore, no vapor blocks are created by water and the preferred solution.

I claim:

1. A method of cleaning and lubricating a detached dental-medical device during a heating sterilization process which comprises:
a. Partial immersing said device in a solution comprising
i. About 50% to about 85% by volume of water
ii. About 5% to about 22% by volume of glycerin
iii. About 0.05% to about 0.20% by volume of chlorhexidine gluconate
iv. About 3% to about 15% by volume of alcohol

2. A method of cleaning and lubricating a detached device partially immersed in a preferred solution that is contained in a sealed heat sterilizable container during the process of heat sterilization.

3. A method of cleaning and lubricating the device's external and internal parts within the sealed heat sterilizable container when the preferred solution contacts the device's parts.

4. A method of cleaning and lubricating the detached device within the sealed heat sterilizable container when the preferred solution begins to boil and then vaporizes to penetrate the internal and external moveable parts and conduits.

5. The miscible property of the preferred solution and water within the handpiece allows the preferred solution to vaporize any remaining water in the device's parts or conduits.

6. The preferred solution vaporizes before water vaporizes. This allows the preferred solution's vapors to penetrate the water.
